(19) United States
(12) Patent Application Publication
(10) Pub. No.: US 2026/0013735 A1
(43) Pub. Date: Jan. 15, 2026

(54) PHYSIOLOGIC MONITORING FOR ISSUES INVOLVING THE PERICARDIUM

(71) Applicant: Cardiac Pacemakers, Inc., St Paul, MN (US)

(72) Inventors: Victoria A. Averina, Shoreview, MN (US); Matthew J. Miller, Stillwater, MN (US); Jonathan B. Shute, Minnetonka, MN (US); Pramodsingh H. Thakur, Woodbury, MN (US)

(21) Appl. No.: 19/234,788

(22) Filed: Jun. 11, 2025

Related U.S. Application Data
(60) Provisional application No. 63/669,113, filed on Jul. 9, 2024.

(51) Int. Cl. A61B 5/0205; A61B 5/00; A61B 5/08; A61B 5/11; A61B 5/1455; A61B 5/355; G16H 40/63

(52) U.S. Cl. CPC A61B 5/0205; A61B 5/08; A61B 5/1116; A61B 5/1118; A61B 5/14551; A61B 5/355; A61B 5/7264; A61B 5/7282; A61B 5/7285; A61B 5/746; G16H 40/63; A61B 2562/0219

(57) ABSTRACT

Systems, computer-readable media, and methods for detecting a first indicator of a pericardium-related issue based, at least in part, on first heart sounds measurements; detecting a second indicator of the pericardium-related issue based, at least in part, on one or more of the following: posture, restlessness, respiratory activity, cardiac activation signals, oxygen saturation, and second heart sounds measurements; and generating an alert in response to detecting the first indicator and the second indicator.

### CROSS REFERENCE TO RELATED APPLICATION

This application claims priority to Provisional Application No. 63/669,113, filed Jul. 9, 2024, which is herein incorporated by reference in its entirety.

### TECHNICAL FIELD

Instances of the present disclosure relate to medical devices and systems for sensing physiological parameters that indicate issues involving the pericardium.

### BACKGROUND

An accumulation of fluid in the intra-pericardial space surrounding the heart may result in an increase in pressure in the intra-pericardial space, indicating the occurrence of cardiac tamponade. “Tamponade” means obstruction of blood flow due to a constriction of a blood channel caused by an outside force, such as overpressure acting on the heart wall. Blood is prevented from entering the heart from the veins due to increased pressure in the intra-pericardial space, resulting in a lowering of blood pressure and tachycardia.

### SUMMARY

In Example 1, a method includes detecting a first indicator of a pericardium-related issue based, at least in part, on first heart sounds measurements. The method further includes detecting a second indicator of the pericardium-related issue based, at least in part, on one or more of the following: posture, restlessness, respiratory activity, cardiac activation signals, oxygen saturation, and second heart sounds measurements. An alert is generated in response to detecting the first indicator and the second indicator, and the alert indicates that the pericardium-related issue is occurring.

In Example 2, the method of Example 1, further includes initiating a detection routine for detecting the first indicator, activating a monitor routine for the detecting the first indicator in response to detecting the first indicator, and initiating the detecting the second indicator after confirming detection of the first indicator during the monitor routine.

In Example 3, the method of Example 2, wherein the detection routine includes periodically evaluating the first heart sounds measurements at a first frequency, wherein the monitor routine includes periodically evaluating another set of heart sounds measurements at a second frequency that is greater than the first frequency.

In Example 4, the method of any of Examples 1-3, wherein the first indicator is pericardial friction rub.

In Example 5, the method of Example 4, wherein the pericardial friction rub is detected based on a frequency level of the first heart sounds measurements.

In Example 6, the method of Example 5, wherein the frequency level is greater than a baseline frequency level during atrial contraction, ventricular systole, and ventricular diastole.

In Example 7, the method of any of Examples 1-6, wherein the second indicator is based on posture, which is based on outputs of a posture sensor, wherein the outputs indicate an increase in leaning forward during periods of inactivity.

In Example 8, the method of any of Examples 1-6, wherein the second indicator is based on activity, which is based on outputs of an activity sensor, wherein the outputs indicate an increase in restlessness.

In Example 9, the method of any of Examples 1-6, wherein the second indicator is based on the second heart sounds measurements, wherein the second heart sounds measurements indicate hypotension and/or muffled heart sounds.

In Example 10, the method of Example 9, wherein the first heart sounds measurements and the second heart sounds measurements are measured by a single accelerometer.

In Example 11, the method of any of Examples 1-6, wherein the second indicator is based on cardiac activation signals, which is based on outputs of an electrocardiogram sensor, wherein the outputs indicate T-wave inversions.

In Example 12, the method of any of Examples 1-6, wherein the second indicator is based on oxygen saturation, which is based on outputs of a pulse oximetry sensor, wherein the outputs indicate respiratory variability in an oxygen saturation waveform.

In Example 13, a computer program product comprising instructions to cause one or more processors to carry out the steps of the method of Examples 1-12.

In Example 14, a computer-readable memory having stored thereon the computer program product of Example 13.

In Example 15, a medical device or a medical system comprising the computer-readable memory of Example 14.

In Example 16, one or more non-transitory computer-readable media have computer-executable instructions embodied thereon. When executed, the instructions cause one or more computing devices to carry out an operation including: detecting a first indicator of a pericardium-related issue based, at least in part, on first heart sounds measurements; detecting a second indicator of the pericardium-related issue based, at least in part, on one or more of the following: posture, restlessness, respiratory activity, cardiac activation signals, oxygen saturation, and second heart sounds measurements; and generating an alert that the pericardium-related issue is occurring in response to detecting the first indicator and the second indicator.

In Example 17, the media of Example 16, the operation further includes: initiating a detection routine for detecting the first indicator; in response to detecting the first indicator, activating a monitor routine for the detecting the first indicator; and after confirming detection of the first indicator during the monitor routine, initiating the detecting the second indicator.

In Example 18, the media of Example 17, wherein the detection routine includes periodically evaluating the first heart sounds measurements at a first frequency, wherein the monitor routine includes periodically evaluating another set of heart sounds measurements at a second frequency that is greater than the first frequency.

In Example 19, the media of Example 16, wherein the first indicator is pericardial friction rub.

In Example 20, the media of Example 19, wherein the pericardial friction rub is detected based on a frequency level of the first heart sounds measurements.

In Example 21, the media of Example 20, wherein the frequency level is greater than a baseline frequency level during atrial contraction, ventricular systole, and ventricular diastole.

In Example 22, the media of Example 16, wherein the second indicator is based on posture, which is based on outputs of a posture sensor, wherein the outputs indicate an increase in leaning forward during periods of inactivity.

In Example 23, the media of Example 16, wherein the second indicator is based on activity, which is based on outputs of an activity sensor, wherein the outputs indicate an increase in restlessness.

In Example 24, the media of Example 16, wherein the second indicator is based on the second heart sounds measurements, wherein the second heart sounds measurements indicate hypotension and/or muffled heart sounds.

In Example 25, the method of Example 24, wherein the first heart sounds measurements and the second heart sounds measurements are measured by a single accelerometer.

In Example 26, the media of Example 16, wherein the second indicator is based on cardiac activation signals, which is based on outputs of an electrocardiogram sensor, wherein the outputs indicate T-wave inversions.

In Example 27, the media of Example 16, wherein the second indicator is based on oxygen saturation, which is based on outputs of a pulse oximetry sensor, wherein the outputs indicate respiratory variability in an oxygen saturation waveform.

In Example 28, a method includes detecting a first indicator of a pericardium-related issue based, at least in part, on first heart sounds measurements; detecting a second indicator of the pericardium-related issue based, at least in part, on one or more of the following: posture, restlessness, respiratory activity, cardiac activation signals, oxygen saturation, and second heart sounds measurements; and generating an alert that the pericardium-related issue is occurring in response to detecting the first indicator and the second indicator.

In Example 29, the method of Example 28, further including: initiating a detection routine for detecting the first indicator; in response to detecting the first indicator, activating a monitor routine for the detecting the first indicator; and after confirming detection of the first indicator during the monitor routine, initiating the detecting the second indicator.

In Example 30, the method of Example 29, wherein the detection routine includes periodically evaluating the first heart sounds measurements at a first frequency, wherein the monitor routine includes periodically evaluating another set of heart sounds measurements at a second frequency that is greater than the first frequency.

In Example 31, the method of Example 28, wherein the first indicator is pericardial friction rub.

In Example 32, the method of Example 31, wherein the pericardial friction rub is detected based on a frequency level of the first heart sounds measurements.

In Example 33, the method of Example 32, wherein the frequency level is greater than a baseline frequency level during atrial contraction, ventricular systole, and ventricular diastole.

In Example 34, the method of Example 32, wherein the second indicator is based on the second heart sounds measurements, wherein the second heart sounds measurements indicate hypotension and/or muffled heart sounds.

In Example 35, a system includes a first sensor configured to detect a first indicator of a pericardium-related issue based, at least in part, on first heart sounds measurements; a second sensor programmed to detect a second indicator of the pericardium-related issue based, at least in part, on one or more of the following: posture, restlessness, respiratory activity, cardiac activation signals, oxygen saturation, and second heart sounds measurements; and a computing device programmed to generate an alert that the pericardium-related issue is occurring in response to detecting the first indicator and the second indicator. In certain instances, the first sensor and the second sensor are part of a single medical device, which may be implanted or positioned external to a patient. In certain instances, the first sensor and the second sensor are part of separate medical devices. In certain instances, the first sensor and the second sensor are both accelerometers.

While multiple instances are disclosed, still other instances of the present disclosure will become apparent to those skilled in the art from the following detailed description, which shows and describes illustrative instances of the invention. Accordingly, the drawings and detailed description are to be regarded as illustrative in nature and not restrictive.

### BRIEF DESCRIPTION OF THE DRAWINGS

FIG. 1 is a schematic illustration depicting an illustrative medical system, in accordance with certain instances of the present disclosure.

FIG. 2 shows block diagram of a medical device and various components of the medical device, in accordance with certain instances of the present disclosure.

FIG. 3 is a block diagram of a method, in accordance with certain instances of the present disclosure.

FIG. 4 is a block diagram of a method, in accordance with certain instances of the present disclosure.

FIG. 5 is a block diagram depicting an illustrative computing device, in accordance with certain instances of the present disclosure.

While the invention is amenable to various modifications and alternative forms, specific instances have been shown by way of example in the drawings and are described in detail below. The intention, however, is not to limit the invention to the particular instances described. On the contrary, the invention is intended to cover all modifications, equivalents, and alternatives falling within the scope of the invention as defined by the appended claims.

### DETAILED DESCRIPTION

Features of the present disclosure relate to medical devices and systems for sensing physiological parameters that indicate issues involving the pericardium. One type of pericardium issue involves accumulation of fluid (e.g., blood) in the intra-pericardial space (e.g., the space between the outer pericardial fibrous layer and the external surface of the heart), which can result in increased pressure in the intra-pericardial space. Another type of pericardium issue involves a lead protruding through a wall of the heart and into the intra-pericardial space, which can cause fluid to pass into the intra-pericardial space.

Certain instances of the present disclosure involve approaches for monitoring and detecting various indicators of pericardium-related issues. Early detection of pericardium-related issues can help reduce the chance that the issues lead to a more severe event.

### Indicators of Pericardium-Related Issues

When a patient is experiencing a pericardium-related issue, the patient may experience one or more symptoms that—individually or collectively—may indicate the occurrence of a pericardium-related issue. These symptoms can be detected via various measurable indicators that suggest that a pericardium-related issue is occurring.

The description below first describes various symptoms and indicators of pericardium-related issues. Next, the description outlines various devices and components that can be used to monitor and detect the indicators. The description then provides approaches for prioritizing certain indicators or assessing various indicators to determine that a pericardium-related issue is likely occurring.

One symptom includes a sudden onset of chest pain, which improves when leaning forward and worsens when lying down. An indicator of this symptom is the patient's posture. The patient's posture can be monitored over time to determine whether the patient tends to lean forward an abnormal amount of time. This indicator can be monitored using a posture sensor (described in more detail below). The outputs of the posture sensor can be used to detect that the patient is experiencing the symptom. For example, the outputs of the posture sensor can be compared to a threshold or compared to a baseline dataset to detect an increase in leaning forward during inactivity and/or avoidance of lying down.

Another symptom includes restlessness. An indicator of this symptom is the patient's activity and/or posture. The patient's activity and/or posture can be monitored over time to determine whether the patient has less frequent periods of resting activity and/or more frequent changes in posture. This indicator can be monitored using an activity sensor (described in more detail below) and/or a posture sensor. The outputs of the one or more sensors can be used to detect that the patient is experiencing the symptom. For example, the outputs of the sensors can be compared to a threshold or compared to a baseline dataset to detect a decrease in periods of rest and/or an increase in changes in posture.

Another symptom includes palpitations. An indicator of this symptom is the patient's cardiac electrical activation activity. The patient's cardiac electrical activation activity can be monitored over time to determine whether the patient is experiencing palpitations. This indicator can be monitored using an electrocardiogram (ECG) sensor (described in more detail below). The outputs of the ECG sensor can be used to detect that the patient is experiencing the symptom. For example, the outputs of the ECG sensor can be compared to a threshold, compared to a baseline dataset, and/or processed by a computing tool (e.g., a machine learning algorithm) to detect the occurrence of palpitations.

Another symptom includes shortness of breath. An indicator of this symptom is the patient's respiratory activity, in particular an increase in the patient's rapid shallow breathing index (RSBI) and/or respiratory rate (RR). The patient's respiratory activity can be monitored over time to determine whether the patient is experiencing shortness of breath. This indicator can be monitored using various types of sensors such as an oxygen saturation (SpO2) sensor (described in more detail below). The outputs of the sensor can be used to detect that the patient is experiencing the symptom. For example, the outputs of the sensor can be used to determine the patient's RSBI (e.g., a ratio of tidal volume (TV) in liters to RR in breaths/minute: RSBI=TV/RR) and/or RR. The RSBI and/or RR can be compared to a threshold and/or compared to a baseline dataset to detect the occurrence of shortness of breath.

Another symptom includes dizziness. An indicator of this symptom is the patient's activity and/or posture. The patient's activity and/or posture can be monitored over time to determine whether the patient has a larger variability in small posture changes. This indicator can be monitored using an activity sensor and/or a posture sensor. The outputs of the one or more sensors can be used to detect that the patient is experiencing the symptom. For example, the outputs of the sensors can be compared to a threshold or compared to a baseline dataset to detect an increase in variability in small posture changes.

Another symptom includes syncope (e.g., a brief loss of consciousness and muscle control caused by a temporary decrease in blood flow to the brain). There are several indicators of this symptom and, therefore, several ways to monitor and detect the occurrence of syncope. One approach uses an ECG sensor to monitor the patient's cardiac electrical activation activity. The outputs of the ECG sensor can be used to detect that the patient is experiencing syncope.

Another symptom includes altered mental status. In certain instances, this can be self-monitored by the patient or a caregiver. One approach of self-monitoring involves the patient or a caregiver providing input to a software application (e.g., a computer application, a mobile phone application). For example, when or after the patient has experienced an altered mental status, the patient or a caregiver can press a button, icon, etc., in the software application to record the experience.

Another indicator of pericardium-related issues is based on the patient's heart sounds (which are described in more detail below). The patient's heart sounds can be measured by a heart sounds sensor (described below). In one example instance, a decrease in S3 amplitude is an indicator of a pericardium-related issue. The decrease can result from excessive pericardial fluid dampening the S3 heart sound. In another example instance, friction between inflamed pericardial layers causes a high-pitched, scratchy sound. This heart sound may be louder during inspiration and may be triphasic (e.g., audible in atrial contraction, ventricular systole, and ventricular diastole). In another example, a lead protruding through a wall of the heart can cause a high-pitched heart sound. The patient's heart sounds can be comparted to a threshold (e.g., an amplitude-based threshold) and/or compared to a baseline dataset to detect the occurrence of dampened S3 heart sounds, pericardial friction rub, and/or lead protrusion.

Another symptom includes the patient's blood oxygen content. In particular, an increased variability in a pulse-oximetry waveform can be an indicator of a pericardium-related issue. For example, instead of the pulse-oximetry waveform having a normal, defined shape; variability in the waveform will be characterized by abnormal shapes or modulation in the waveform. Blood oxygen content can be measured by an oxygen saturation (SpO2) sensor. Outputs of the sensor can be compared to a threshold and/or compared to a baseline dataset to detect the occurrence of an increase in variability in a pulse-oximetry waveform.

One set of symptoms includes what is sometimes referred to as Beck's triad, which includes the occurrence of three different symptoms: hypotension, jugular distension, and muffled heart sounds.

Hypotension can be monitored and detected using multiple approaches. In certain instances, a patient's blood pressure and blood pressure trends over time can be measured directly with a blood pressure sensor. Another approach utilizes heart sounds, which can be used to monitor and detect a decline in S2 heart sounds (e.g., daily S2, an increased reduction of S2 synced with respiratory rate), which indicates a reduction in blood pressure. Another approach utilizes the patient's pulse rate. Another approach utilizes cardiac impedance amplitude (e.g., weak pulse, reduced stroke volume). Another approach utilizes an increase in respiratory variability. Another approach utilizes oxygen saturation. The outputs of the one or more sensors can be used to detect that the patient is experiencing hypotension. In certain instances, a decrease of 10 mmHg or more with inspiration is used to detect hypotension.

Jugular distension can be self-monitored by the patient or a caregiver. One approach of self-monitoring involves the patient or a caregiver providing input to a software application. For example, when or after the patient is experiencing jugular distension, the patient or a caregiver can press a button, icon, etc., in the software application to record the experience.

Certain muffled heart sounds can be another indicator of pericardium-related issues. In general, a reduction in amplitude of heart sounds suggest muffled heart sounds. One particular type of reduction that can be monitored is a reduction in amplitude of S1 heart sounds.

Another indicator of pericardium-related issues is the patient's cardiac electrical activation activity, which can be sensed by an ECG sensor. The patient's ECG data can evolve as a pericardium-related issue arises. In certain instances, the ECG data evolves through four stages. First, there is a diffuse, concave-up ST-segment elevation, which in certain instances is accompanied by PR-segment elevation in lead AVR. Second, there is normalization of ST and PR segment changes within a week. Third, the normalization is followed by widespread T-wave inversions. Fourth, the inversions are followed by T-wave normalization. This evolution of cardiac electrical activation activity can be detected by analysis of the patient's ECG data. For example, a medical device can be programmed with various thresholds to detect the occurrence of one or more stages mentioned herein.

The indicators described herein can be monitored and detected using one or more medical devices described below.

### Medical System

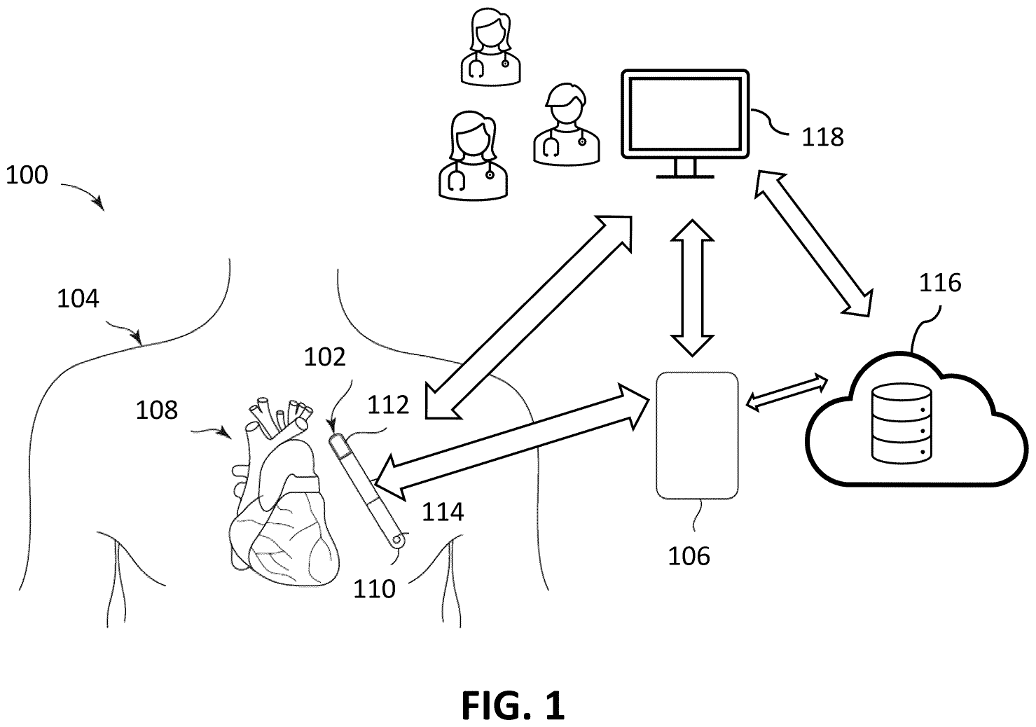

FIG. 1 is a schematic illustration of a system 100 including an implantable medical device (IMD) 102 implanted within a patient's body 104 and an external medical device (EMD) 106 coupled to the patient.

The IMD 102 may be implanted subcutaneously within an implantation location or pocket in the patient's chest or abdomen and may be configured to monitor (e.g., sense and/or record) physiological parameters associated with the patient's heart 108. The IMD 102 may be an implantable cardiac monitor (e.g., an implantable diagnostic monitor, an implantable loop recorder) configured to record physiological parameters such as, for example, the indicators mentioned above. For purposes of illustration, and not of limitation, various instances of devices that may be used to monitor and/or record physiological parameters in accordance with the present disclosure are described herein in the context of IMDs that may be implanted. However, the IMD 102 may include any type of IMD, any number of different components of an implantable system, and/or the like having a housing and being configured to be implanted in a patient's body 104. For example, the IMD 102 may include a control device, a monitoring device, a pacemaker, an implantable cardioverter defibrillator (ICD), a cardiac resynchronization therapy (CRT) device and/or the like, and may be an implantable medical device known in the art or later developed, for providing therapy and/or diagnostic data about the patient's body. In various instances, the IMD 102 may include both defibrillation and pacing/CRT capabilities (e.g., a CRT-D device). As shown, the IMD 102 may include a housing 110 having two electrodes 112 and 114 coupled thereto.

The EMD 106 can be a device such as a patch, wearable device (e.g., watch, ring, bracelet), strap, mobile phone, and the like.

The medical devices may communicate through wireless links. For example, the medical devices can include communication devices such as an antenna, which can transmit and/or receive wireless signals. The medical devices can be in direct or indirect communication with a computing system 116 (e.g., laptop computer, desktop computer, server) and/or to another computing system 118 with a display on which users (e.g., patients, physicians, technicians) can review data generated by the medical device 100.

In certain instances, the medical devices are programmed to detect indicators based on the physiological data sensed by the medical devices. For example, the medical devices can compare the physiological data to various programmed parameters (e.g., thresholds, baselines) and, if one or more parameters are met (e.g., thresholds breached, increase/decrease from baselines), the medical devices can determine that a potential health event has occurred.

In certain instances, the recorded physiological data (and associated metadata) may be downloaded from the medical devices periodically or on command. When an indicator is detected (based on one or more programmed parameters), the medical devices can begin to store data associated with the detected indicator to local memory. In some instances, the medical devices are configured to repeatedly delete a certain amount of historical sensed physiological data until an indicator is detected. For example, the medical devices may continuously delete historical data (that is not associated with a detected indicator) after expiration of a rolling period of time (e.g., 10-20 seconds) to preserve memory capacity. However, once an indicator is detected, the medical devices can begin storing physiological data.

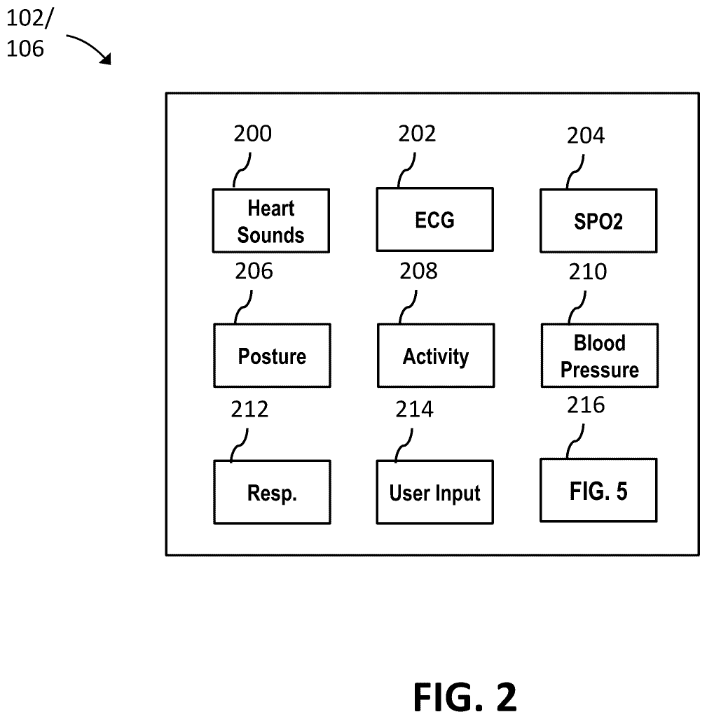

FIG. 2 shows a block diagram of components that can be incorporated into the IMD 102 and/or the EMD 106. For example, some of the components shown in FIG. 2 can be incorporated into the IMD 102 while other components shown in FIG. 2 can be incorporated into the EMD 106. In some instances, the IMD 102 and the EMD 106 may have the same type of component (e.g., both devices may have their own ECG sensor or other type of sensor). Further, some components shown in FIG. 2 may be incorporated into a third device or may be standalone components (e.g., standalone sensors attached to the patient). Not all of the of components shown in FIG. 2 may be present in a given medical system.

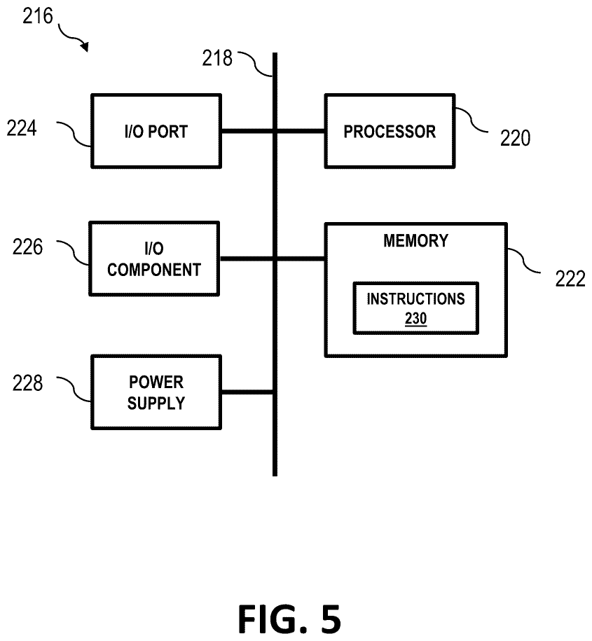

The components shown in FIG. 2 include a heart sounds sensor 200, an ECG sensor 202, an oxygen saturation (SpO2) sensor 204, a posture sensor 206, an activity sensor 208, a blood pressure sensor 210, a respiratory sensor 212, user input device 214, and a computing device 216 (which is described in more detail in connection with FIG. 5). The various sensors may be implantable (e.g., part of the IMD 102) or positioned external to the patient (e.g., part of the EMD 106).

The heart sounds sensor 200 is configured to detect heart sounds. The opening and closing of valves—as well as the flow of blood through the heart—produce acoustic signals known as heart sounds. Heart sounds may be measured and used, for example, to indicate the heart's mechanical activities. A heart sound can include audible and inaudible mechanical vibrations caused by cardiac activity that can be sensed with an acceleration sensor (e.g., an accelerometer). As such, the heart sounds sensor 200 can be an acceleration sensor such as a 1-D accelerometer or a multi-axis accelerometer.

There are different clinical categories of heart sounds. For example, the S1heart sound refers to the first heart sound of a cardiac cycle, S2 refers to the second heart sound, S3 refers to the third heart sound, and S4 refers to the fourth heart sound. S1 is known to be indicative of, among other things, mitral valve closure, tricuspid valve closure, and aortic valve opening. S2 is known to be indicative of, among other things, aortic valve closure and pulmonary valve closure. S3 is known to be a ventricular diastolic filling sound often indicative of certain pathological conditions including heart failure. And S4 is known to be a ventricular diastolic filling sound resulted from atrial contraction and is usually indicative of pathological conditions.

The ECG sensor 202 is configured to detect electrical impulses generated by heart muscles. The ECG sensor 202 can include electrically conductive electrodes that are used to detect the electrical impulses.

The SpO2 sensor 204 is configured to detect oxygen saturation within the patient's blood. In certain instances, the output of the SpO2 sensor 204 is a photoplethysmography (PPG) signal that indicates, over time, a person's blood oxygen content. In instances where the SpO2 sensor 204 is part of the EMD 106, the SpO2 sensor 204 can be placed on a person's body part (e.g., finger, ear lobe) so that light is passed through the body part to an optical sensor. The person's blood oxygen content (or oxygen saturation) can be estimated based on how the light is absorbed as the light passes through blood flowing in the body part. In instances where the SpO2 sensor 204 is part of the IMD 102, the SpO2 sensor 204 can include an optical module with one or more emitters (e.g., one or more light sources such as light-emitting diodes) and one or more detectors (e.g., one or more light sensors such as photodetectors or another type of light sensor). The emitters emit light towards a patient's tissue, and at least some light will be reflected back (e.g., backscattered light) to be sensed by the detectors. The sensed backscattered light can be used by the SpO2 sensor 204 to measure the patient's blood oxygen content (e.g., SpO2).

The posture sensor 206 can measure a patient's posture such as whether a patient is in an upright position, a supine position, a prone position, on his or her left or right side, or if the patient is in a tilted position. In certain instances, the posture sensor 206 includes a multi-axis accelerometer.

The activity sensor 208 can measure a patient's activity such as whether the patient is at rest, walking, running, etc. In certain instances, the activity sensor 208 is a momentum sensor, gyroscope, accelerometer (e.g., a 1-D accelerometer or a multi-axis accelerometer). In certain instances, the posture sensor 206 and the activity sensor 208 are the same sensor.

The blood pressure sensor 210 is configured to measure the patient's blood pressure, and the respiratory sensor 212 is configured to measure the patient's respiratory activity such as respiratory rate. The user input device 214 can be a physical button on the EMD 106 and/or a button, icon, etc., in a software application (e.g., an application on a smartphone).

### Methods

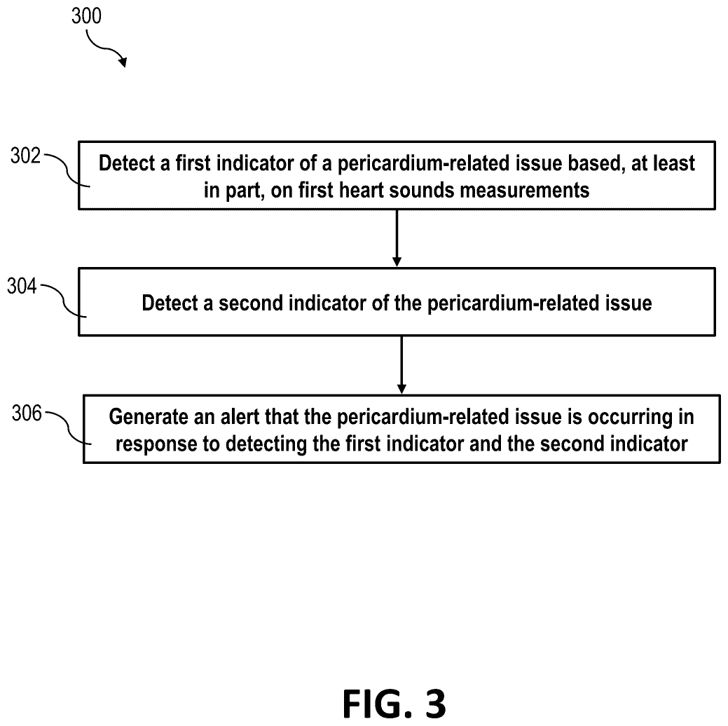

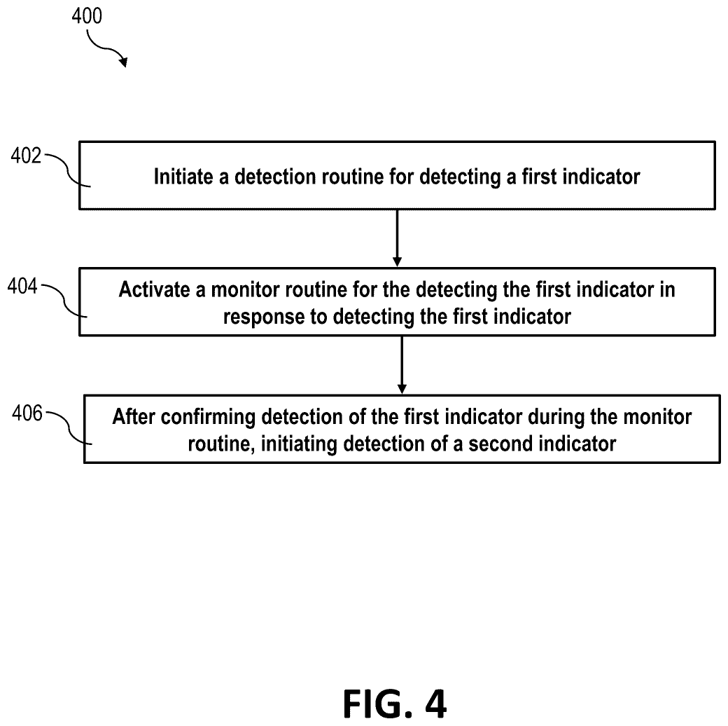

FIGS. 3 and 4 outline approaches for prioritizing the monitoring of certain indicators and/or assessing a collection of indicators to determine that a pericardium-related issue is likely occurring. The method of FIG. 3 is focused on monitoring and detecting at least two indicators to determine a risk of a pericardium-related issue. The method of FIG. 4 is focused on detecting one indicator initially and then starting to monitor a second indicator to confirm a risk that a pericardium-related issue is occurring.

FIG. 3 shows a block diagram of a method 300 for use with the IMD 102 and/or the EMD 106. The method 300 includes detecting a first indicator of a pericardium-related issue based, at least in part, on first heart sounds measurements (block 302 in FIG. 3). As noted above, heart sounds can be used to detect a variety of indicators of a pericardium-related issue. Examples include a decrease in S3 amplitude (e.g., indicating excessive pericardial fluid dampening the S3 heart sound), a high-pitched heart sound (e.g., indicating friction between inflamed pericardial layers or a lead protruding through a wall of the heart), a decline in S2 heart sounds (e.g., indicating a reduction in blood pressure), and a reduction in amplitude of S1 heart sounds (e.g., indicating muffled heart sounds). As noted above, the heart sounds can be detected by a heart sounds sensor (e.g., an acceleration sensor) that is part of the IMD 102 and/or the EMD 106. The output of the heart sounds sensor can be inputted into a computing device, and the computing device can be programmed to detect one or more of the heart sounds indicators noted herein. For example, the computing device can be programmed to compare the output of the heart sounds sensor to a threshold (e.g., amplitude) or a baseline (e.g., amplitude). In certain instances, the output of the heart sounds sensor is filtered (e.g., bandpass filtered, high-pass filtered, low-pass filtered) to attenuate frequencies that are likely not indicative of heart sounds. As such, the heart sounds sensor and/or the computing device can include a digital filter.

The method 300 further includes detecting a second indicator of the pericardium-related issue based, at least in part, on one or more of the following: posture, restlessness, respiratory activity, cardiac activation signals, oxygen saturation, and second heart sounds measurements (block 304 in FIG. 3). As noted above, the second indicator can be detected by one or more sensors that are part of the IMD 102 and/or the EMD 106. The data monitored for the first indicator and the data monitored for the second indicator can be time-synched.

In certain instances, the heart sounds sensor and the sensor for the second indicator are part of the same device. In certain instances, the heart sounds sensor and the second indicator are both acceleration sensors (or the same acceleration sensor).

The output of the sensor can be inputted into a computing device, and the computing device can be programmed to detect the second indicator.

The method 300 further includes generating an alert that the pericardium-related issue is occurring in response to detecting the first indicator and the second indicator (block 306 in FIG. 3). The alert can be generated by any device within a medical system, including one of the medical devices or a separate computing device/system. The alert can be transmitted to the patient's physician (e.g., clinic, physician group) via the device/system that generates the alert. In certain instances, once two indicators are detected, the alert is generated. In other instances, the alert is generated once three, four, five, etc. indicators are detected (e.g., via a voting algorithm that requires a minimum number of votes before an alert is generated). In other instances, the alert is generated once a confidence level is reached. The confidence level can be based on a voting algorithm, a value calculated by weighting the detected indicators (e.g., by giving more weight to the indicators more likely to suggest a pericardium-related issue), or other approaches that take into account multiple detected indicators (or the lack of detecting certain indicators).

In certain instances, the first indicator and the second indicator are both monitored using the same sensor (e.g., an acceleration sensor), and the first indicator and the second indicator are detected using the same computing device. In certain instances, once the first indicator and the second indicator are detected, a third indicator (e.g., based on ECG data) can be used to confirm that the pericardium-related issue is occurring.

FIG. 4 shows a block diagram of a method 400 for use with the IMD 102 and/or the EMD 106. The method 400 of FIG. 4 includes initiating different routines for power-efficient monitoring and detection of indicators.

The method 400 includes initiating a detection routine for detecting a first indicator (block 402 in FIG. 4). In certain instances, the detection routine includes periodically evaluating an indicator at a first frequency. Periodically evaluating can mean that the first indicator is monitored for limited periods of time to reduce the amount of power required to operate the given sensor and the power required to operate a computing device to compare the sensor outputs to a threshold, baseline, etc. For example, the first indicator may be initially monitored for a predetermined amount of time (and at predetermined times) each day, each hour, etc. As one specific example, the first indicator could be monitored once every 23 hours (e.g., for one hour each day and at a different starting time). The first indicator can be any of the indicators described herein. In certain instances, the first indicator is an indicator selected because it is the most efficient (e.g., most power-efficient) indicator to measure.

The method 400 further includes activating a monitor routine for the detecting the first indicator in response to detecting the first indicator (block 404 in FIG. 4). In the monitor routine, the first indicator can be periodically evaluated at a second frequency that is greater than the first frequency. In other words, the first indicator can be measured more often and/or for longer periods of time compared to the detection routine.

The method 400 further includes initiating detection a second indicator after confirming detection of the first indicator during the monitor routine (block 406 in FIG. 4). In certain instances, the process of monitoring and detecting the second indicator may require using more power compared to monitoring and detecting the first indicator. If the second indicator is detected, it can be determined that a pericardium-related issue is likely to be occurring. The second indicator can be any of the indicators described herein.

Using the approaches outlined in FIGS. 3 and 4, multiple indicators can be monitored and detected to determine that a pericardium-related issue is likely to be occurring. The approaches outlined in FIGS. 3 and 4 are not mutually exclusive and can be used together to monitor and detect indicators of pericardium-related issues.

### Computing Device

FIG. 5 shows additional details of the computing device 216 (which was initially shown in FIG. 2). FIG. 5 is a block diagram depicting an illustrative computing device 216, in accordance with instances of the disclosure. The computing device 216 may include any type of computing device suitable for implementing aspects of instances of the disclosed subject matter. Examples of computing devices include specialized computing devices or general-purpose computing devices such as workstations, servers, laptops, desktops, tablet computers, hand-held devices, smartphones, general-purpose graphics processing units (GPGPUs), and the like. Each of the various components shown and described in the Figures can contain their own dedicated set of computing device components shown in FIG. 5 and described below. For example, the medical devices, servers, and remote computers can each include their own set of components shown in FIG. 5 and described below.

In instances, the computing device 216 includes a bus 218 that, directly and/or indirectly, couples one or more of the following devices: a processor 220, a memory 222, an input/output (I/O) port 224, an I/O component 226, and a power supply 228. Any number of additional components, different components, and/or combinations of components may also be included in the computing device 216.

The bus 218 represents what may be one or more busses (such as, for example, an address bus, data bus, or combination thereof). Similarly, in instances, the computing device 216 may include a number of processors 220, a number of memory components 222, a number of I/O ports 224, a number of I/O components 226, and/or a number of power supplies 228. Additionally, any number of these components, or combinations thereof, may be distributed and/or duplicated across a number of computing devices.

In instances, the memory 222 includes computer-readable media in the form of volatile and/or nonvolatile memory and may be removable, nonremovable, or a combination thereof. Media examples include random access memory (RAM); read only memory (ROM); electronically erasable programmable read only memory (EEPROM); flash memory; optical or holographic media; magnetic cassettes, magnetic tape, magnetic disk storage or other magnetic storage devices; data transmissions; and/or any other medium that can be used to store information and can be accessed by a computing device. In instances, the memory 222 stores computer-executable instructions 230 for causing the processor 220 to implement aspects of instances of components discussed herein and/or to perform aspects of instances of methods and procedures discussed herein. The memory 222 can comprise a non-transitory computer readable medium storing the computer-executable instructions 230.

The computer-executable instructions 230 may include, for example, computer code, machine-useable instructions, and the like such as, for example, program components capable of being executed by one or more processors 220 (e.g., microprocessors) associated with the computing device 216. Program components may be programmed using any number of different programming environments, including various languages, development kits, frameworks, and/or the like. Some or all of the functionality contemplated herein may also, or alternatively, be implemented in hardware and/or firmware.

According to instances, for example, the instructions 230 may be configured to be executed by the processor 220 and, upon execution, to cause the processor 220 to perform certain processes. In certain instances, the processor 220, memory 222, and instructions 230 are part of a controller such as an application specific integrated circuit (ASIC), field-programmable gate array (FPGA), and/or the like. Such devices can be used to carry out the functions and steps described herein.

The I/O component 226 may include a presentation component configured to present information to a user such as, for example, a display device, a speaker, a printing device, and/or the like, and/or an input component such as, for example, a microphone, a joystick, a satellite dish, a scanner, a printer, a wireless device, a keyboard, a pen, a voice input device, a touch input device, a touch-screen device, an interactive display device, a mouse, and/or the like.

The devices and systems described herein can be communicatively coupled via a network, which may include a local area network (LAN), a wide area network (WAN), a cellular data network, via the internet using an internet service provider, and the like.

Aspects of the present disclosure are described with reference to flowchart illustrations and/or block diagrams of methods, devices, systems and computer program products. It will be understood that each block of the flowchart illustrations and/or block diagrams, and combinations of blocks in the flowchart illustrations and/or block diagrams, can be implemented by computer program instructions.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. One or more non-transitory computer-readable media having computer-executable instructions embodied thereon, the instructions for execution to cause one or more computing devices to carry out an operation comprising:
detecting a first indicator of a pericardium-related issue based, at least in part, on first heart sounds measurements;
detecting a second indicator of the pericardium-related issue based, at least in part, on one or more of the following: posture, restlessness, respiratory activity, cardiac activation signals, oxygen saturation, and second heart sounds measurements; and
generating an alert that the pericardium-related issue is occurring in response to detecting the first indicator and the second indicator.

2. The media of claim 1, the operation further comprising:
initiating a detection routine for detecting the first indicator;
in response to detecting the first indicator, activating a monitor routine for the detecting the first indicator; and
after confirming detection of the first indicator during the monitor routine, initiating the detecting the second indicator.

3. The media of claim 2, wherein the detection routine includes periodically evaluating the first heart sounds measurements at a first frequency, wherein the monitor routine includes periodically evaluating another set of heart sounds measurements at a second frequency that is greater than the first frequency.

4. The media of claim 1, wherein the first indicator is pericardial friction rub.

5. The media of claim 4, wherein the pericardial friction rub is detected based on a frequency level of the first heart sounds measurements.

6. The media of claim 5, wherein the frequency level is greater than a baseline frequency level during atrial contraction, ventricular systole, and ventricular diastole.

7. The media of claim 1, wherein the second indicator is based on posture, which is based on outputs of a posture sensor, wherein the outputs indicate an increase in leaning forward during periods of inactivity.

8. The media of claim 1, wherein the second indicator is based on activity, which is based on outputs of an activity sensor, wherein the outputs indicate an increase in restlessness.

9. The media of claim 1, wherein the second indicator is based on the second heart sounds measurements, wherein the second heart sounds measurements indicate hypotension and/or muffled heart sounds.

10. The method of claim 9, wherein the first heart sounds measurements and the second heart sounds measurements are measured by a single accelerometer.

11. The media of claim 1, wherein the second indicator is based on cardiac activation signals, which is based on outputs of an electrocardiogram sensor, wherein the outputs indicate T-wave inversions.

12. The media of claim 1, wherein the second indicator is based on oxygen saturation, which is based on outputs of a pulse oximetry sensor, wherein the outputs indicate respiratory variability in an oxygen saturation waveform.

13. A method comprising:
detecting a first indicator of a pericardium-related issue based, at least in part, on first heart sounds measurements;
detecting a second indicator of the pericardium-related issue based, at least in part, on one or more of the following: posture, restlessness, respiratory activity, cardiac activation signals, oxygen saturation, and second heart sounds measurements; and
generating an alert that the pericardium-related issue is occurring in response to detecting the first indicator and the second indicator.

14. The method of claim 13, further comprising:
initiating a detection routine for detecting the first indicator;
in response to detecting the first indicator, activating a monitor routine for the detecting the first indicator; and
after confirming detection of the first indicator during the monitor routine, initiating the detecting the second indicator.

15. The method of claim 14, wherein the detection routine includes periodically evaluating the first heart sounds measurements at a first frequency, wherein the monitor routine includes periodically evaluating another set of heart sounds measurements at a second frequency that is greater than the first frequency.

16. The method of claim 13, wherein the first indicator is pericardial friction rub.

17. The method of claim 16, wherein the pericardial friction rub is detected based on a frequency level of the first heart sounds measurements.

18. The method of claim 17, wherein the frequency level is greater than a baseline frequency level during atrial contraction, ventricular systole, and ventricular diastole.

19. The method of claim 17, wherein the second indicator is based on the second heart sounds measurements, wherein the second heart sounds measurements indicate hypotension and/or muffled heart sounds.

20. A system comprising:
a first sensor configured to detect a first indicator of a pericardium-related issue based, at least in part, on first heart sounds measurements;
a second sensor programmed to detect a second indicator of the pericardium-related issue based, at least in part, on one or more of the following: posture, restlessness, respiratory activity, cardiac activation signals, oxygen saturation, and second heart sounds measurements; and
a computing device programmed to generate an alert that the pericardium-related issue is occurring in response to detecting the first indicator and the second indicator.
